(19) United States
(12) Patent Application Publication
(10) Pub. No.: US 2016/0263157 A1
(43) Pub. Date: Sep. 15, 2016

(54) APPLICATION AND PHARMACEUTICAL COMPOSITION OF PREACTIVATED AND DISAGGREGATED SHAPE-CHANGED PLATELETS

(71) Applicants: Yuan-Ji Day, Taoyuan County (TW); Li-Man Hung, New Taipei City (TW); Shiang-Suo Huang, Taipei City (TW); Hsiang-Ruei Liao, Taipei City (TW); Chiou-Mei Lee, Taoyuan County (TW); Jiin-Tarng Liou, Taoyuan County (TW); Fu-Chao Liu, Taoyuan County (TW); Chih-Chieh Mao, Taoyuan County (TW); Polung Yang, Taoyuan County (TW)

(72) Inventors: Yuan-Ji Day, Taoyuan County (TW); Li-Man Hung, New Taipei City (TW); Shiang-Suo Huang, Taipei City (TW); Hsiang-Ruei Liao, Taipei City (TW); Chiou-Mei Lee, Taoyuan County (TW); Jiin-Tarng Liou, Taoyuan County (TW); Fu-Chao Liu, Taoyuan County (TW); Chih-Chieh Mao, Taoyuan County (TW); Polung Yang, Taoyuan County (TW)

(21) Appl. No.: 15/158,345

(22) Filed: May 18, 2016

Related U.S. Application Data
(63) Continuation-in-part of application No. 14/052,755, filed on Oct. 13, 2013.

(51) Int. Cl. A61K 35/19; C12N 5/078; A61K 9/00

(52) U.S. Cl. CPC A61K 35/19; A61K 9/0019; C12N 5/0644; A61K 2035/124

(57) ABSTRACT

Preactivated and disaggregated shape-changed platelets, fixed shape-changed platelets, and a pharmaceutical composition thereof are used for treating acute and emergent inflammatory disease in a dosage of 1×10⁶ to 1×10⁸. SC-PLT is a disaggregated and activated platelet, the secretome from platelet activation has been deprived after SC-PLT preparation but their membrane still preserve cell adhesion molecular and integrin glycoprotein (eg. P-selectin, GP1a, and GPIIb etc.). Therefore, SC-PLTs can trap stromal vascularity inflammatory cells from inflammated and damaged place, and the stromal vascularity inflammatory cells are eliminated through the circulatory system to alleviate inflammation. The fixed shape-changed platelets are able to alleviate inflammation and sustainable for longer storage duration.

### CROSS-REFERENCES TO RELATED APPLICATIONS

This application is a continuation in part of U.S. application Ser. No. 14/052,755, filed Oct. 13, 2013, entitled “APPLICATION AND PHARMACEUTICAL COMPOSITION OF PREACTIVATED AND DISAGGREGATED SHAPE-CHANGED PLATELETS”. The entire teachings of the above application are incorporated herein by reference.

### BACKGROUND OF THE INVENTION

1. Field of the Invention

The present invention relates to shape-changed platelets (SC-PLTs) and a pharmaceutical composition thereof for use in treating acute and emergent inflammatory injuries in a dosage of 1×10⁶ to 2×10⁸.

2. Descriptions of the Related Art

The known platelet-rich plasma (PRP) can be classified into non-activated PRP and activated PRP, and the latter includes PRP hydrogels and emissions or extracts of PRP. PRP hydrogels is fibrinogen released from activated platelets, and it is also used for bone grafts, plastic surgery, dental use and repairing damaged tissues. The emissions or extracts of PRP are rich in growth factors and active proteins, and are considered to promote wound healing and repair damaged bone and joint. The emissions of activated PRP are believed to help repair damaged tissue. However, the related technology, products and patents of the emissions of activated PRP are restricted only in isolation, research and preparation of clottable concentrate of platelet rich in growth factors or related compositions.

Hepatic ischemia-reperfusion (IR) is a topic of great medical and clinical importance, and chronic inflammatory diseases are also injuries of repeated ischemia-reperfusion. The inventors of the subject application indicate that platelets, in addition to a blood clotting function, regulate hepatic inflammation, and prove that shape-changed platelets can reduce acute and emergent inflammatory diseases during hepatic IR.

### SUMMARY OF THE INVENTION

The primary objective of this invention is to provide SC-PLTs and a pharmaceutical composition thereof for use in treating acute and emergent inflammatory diseases in a dosage of 1×10⁶ to 2×10⁸.

Another objective of this invention is to provide fixed-SC-PLTs and a pharmaceutical composition thereof for use in treating acute and emergent inflammatory diseases in a dosage of 1×10⁶ to 2×10⁸.

The fixed-SC-PLTs in a dosage of 1×10⁶ to 2×10⁸ can reduce acute inflammatory injuries, and this proves that the therapeutic effect of the SC-PLTs in reducing acute inflammatory injuries is related to cell surface molecules; and the fixed SC-PLT is sustainable for longer storage duration because it contains no growth factors.

The detailed technology and preferred embodiments implemented for the subject invention are described in the following paragraphs accompanying the appended drawings for people skilled in this field to well appreciate the features of the claimed invention. For the purpose of illustrating the principle of the present invention, the drawings are not necessarily drawn to scale.

### BRIEF DESCRIPTION OF THE DRAWINGS

FIG. 1 is a diagram showing the effect of different dosages of SC-PLTs in treating inflammatory injuries incurred by liver ischemia-reperfusion;

FIG. 2A shows the SC-PLTs used in dermal burn and repair which is measured by a ruler;

FIG. 2B shows the SC-PLTs used in dermal burn and repair, which is detected by hematoxylin and eosin (H&E) Staining;

FIG. 3A shows the SC-PLTs used for increasing viability by reducing inflammatory injuries incurred of sepsis;

FIG. 3B shows the SC-PLTs used in reducing complications of (lung) respiratory failure incurred by sepsis;

FIG. 4A is a schematic diagram showing the SC-PLTs used for increasing viability by reducing damage incurred of ischemic stroke;

FIG. 4B shows the SC-PLTs used for reducing brain infarction incurred by ischemic stroke (n=8, Mean±SE)*, p<0.05;

FIG. 4C shows the SC-PLTs for reducing brain edema incurred by ischemic stroke, the brain water content (%) in the cerebral cortex and striatum (n=8, Mean±SE)*, p<0.05;

FIG. 5 shows the SC-PLTs and fixed-SC-PLTs used for reducing inflammatory injury of liver after 60 minutes ischemia followed by 24 hours reperfusion Mean±SD)*, p<0.05;

FIG. 6A shows that the SC-PLTs are better than Tyrode's buffer in lowering the abdominal inflammatory cells (CD3e+T lymphocyte) in the CLP sepsis mice;

FIG. 6B shows that the SC-PLTs are better than Tyrode's buffer in lowering abdominal CD 11b+monocyte in the CLP sepsis mice; and

FIG. 6C shows that SC-PLTs are better than Tyrode's buffer in lowering abdominal Ly6G+neutrophil in the CLP sepsis mice.

### DETAILED DESCRIPTION OF THE INVENTION

Unless otherwise stated herein, the terms “a (an)”, “the” or the like used in this specification (especially in the Claims hereinafter) shall be understood to encompass both the singular form and the plural form.

The present invention provides shape-changed platelets (SC-PLTs) and a pharmaceutical composition thereof in a dosage of 1×10⁶ to 2×10⁸ for use in treating acute and emergent inflammatory diseases comprising hepatic ischemia-reperfusion inflammatory injuries, sepsis, ischemic stroke, burns, and combinations thereof.

Characteristics of the SC-PLTs in a dosage of 1×10⁶ to 2×10⁸ are as follows. The acute and emergent inflammatory disease is mitigated by activating and transforming the SC-PLT to release cellular adhesion molecules, and the cellular adhesion molecules transfer to the surface of the SC-PLT; the SC-PLTs containing the cellular adhesion molecules are able to adsorb stromal vascularity inflammatory immune cells from inflamed and damaged place, and the inflammatory immune cells are eliminated through the circulatory system. Preparation of SC-PLTs is as follows: an extremely low dose of an agonist is used to activate the non-activated PRP to obtain disaggregated SC-PLTs.

Fixed-SC-PLTs for treating acute and emergent inflammatory diseases in a dosage of 1×10⁶ to 2×10⁸ are provided, wherein the acute and emergent inflammatory diseases comprise hepatic ischemia-reperfusion inflammatory injuries, sepsis, ischemic stroke, burns, and combinations thereof.

Preparation of fixed-SC-PLTs is as follows: fixing the SC-PLTs with 0.5% paraformaldehyde and washing the released growth factors with a buffer, and suspending the SC-PLTs uniformly in the buffer. The fixed-SC-PLTs in a dosage of 1×10⁶ to 2×10⁸ can reduce acute inflammatory injuries, and this proves that the therapeutic effect of SC-PLTs in reducing acute inflammatory injuries is related to cellular factors on cell surface. Because the fixed SC-PLTs in a dosage of 1×10⁶ to 2×10⁸ contain no cellular growth factors, they are sustainable for longer storage duration and mitigate acute inflammatory injuries.

The present invention uses the fixed-SC-PLTs in a dosage of 1×10⁶ to 2×10⁸ for reducing acute inflammatory injuries. The fixed-SC-PLTs are sustainable for longer storage duration compared to PRP. The application of PRP is limited to cosmetic surgery, arthritis, bone graft materials, and repair medication of wounded tissues; however, the SC-PLTs and fixed-SC-PLTs are of more importance and have broader applications including all inflammatory damages, for example, sepsis, ischemic stroke, burns, hepatic ischemia-reperfusion inflammatory injuries, and surgically induced ischemia-reperfusion. The activated SC-PLTs are useful for reducing acute and emergent inflammatory diseases, and they are also useful for development and design of anti-inflammation drugs.

The present invention provides an anti-inflammation pharmaceutical composition for treating acute and emergent inflammatory diseases, comprising SC-PLTs in a dosage of at least 1×10⁶ to 2×10⁸.

### Examples

### A. Preparation of SC-PLT

The SC-PLTs are prepared by the following method:

(a) Male mice are anesthetized and blood is obtained by cardiac puncture. Blood is collected into a sterile tube containing acid citrate dextrose (ACD, 85 mM Sodium citrate, 71 mM Citric acid, and 0.1M dextrose) by ratio of 6:1 (blood:ACD), and then centrifuged at 25° C. and at a maximum speed of 2,300×g for 8 sec per ml. Acceleration and brake speed of the centrifugation are set to 9 and 0, respectively. The upper layer containing platelet-rich plasma (PRP) is harvested to another sterile tube, and then subjected to centrifugation at 2,000×g for 2-3 min per ml (Accel.=9, brake speed=0). Discard the supernatant, and redissolve pellets in 5 ml Tyrode's albumin buffer (134 mM NaCl, 0.34 mM Na₂HPO₄, 2.9 mM KCl, 12 mM NaHCO₃, 20 mM HEPES, pH 7.0, 5 mM glucose, 0.35% (w/v) bovine serum albumin, pH7.0) containing 10 U/mL heparin, and 0.5 μM prostacyclin (PGI2). After 10-min incubation at 37° C. and centrifugation at 2,050×g for 6 min (Accel.=9, brake speed=0), cell pellets are resuspended in 5 ml Tyrode's albumin buffer containing 0.5 μM Prostaglandin I2 (PGI2);

(b) platelet number is counted by a Drew Hemavet 950FS®analyzer (Drew Scientific, Oxford, Conn., USA) and adjusted to a concentration of 2.5˜3×10⁸ platelets/ml with Tyrode's albumin buffer containing 0.02 U/ml apyrase. After 1˜2 min incubation at 25° C., centrifugation, and wash once, the cell pellets are resuspended into Tyrode's albumin buffer. Platelets are activated by thrombin (0.01 U/mL-0.05 U/mL), and tracked the transition of shape-change platelets (SC-PLTs) by platelet aggregometry. The platelet solution is immediately centrifuged, and then pellets are washed, and then resupended in Tyrode's albumin buffer;

(c) conducting a pre-activation process on the platelets using an agonist including adenosine diphosphate (ADP), collagen, thrombin, thrombin receptor agonist peptide (TRAP), reactive oxygen species (ROS) or combinations thereof to obtain the preactivated SC-PLT; and the transforming process of the SC-PLT is as follows: at the pre-activation process the rounded cell membrane is transformed into a pseudopodia form; at this time the preactivated SC-PLT is in a form of proaggregatory suspension particles, and the transmittance of the preactivated SC-PLT decreases to a stable state, and is negative in a blood agglutination, so as to obtain the SC-PLT; wherein when the platelets react with an agonist in a blood agglutination dosage, and then react with fibrinogen, the platelets irreversibly become a polymolecular procoagulant from a transition state of small suspension particles, and the transmittance of the platelets in decreases rapidly and temporarily and then increases abruptly to the top (i.e., blood agglutination); wherein the SC-PLTs are preactivated disaggregated platelets, and the SC-PLTs emit biologically active substances stored in cells, α-granules, dense granules and lysosomes out to cells or transport these substances to the surface of cellular membrane; and the factors transported to the surface of cellular membrane include the cellular adhesion molecule. In one exemplary example the buffer is Tyrode's buffer.

### B. Preparation of Fixed-SC-PLT

The fixed-SC-PLT is prepared by the following method:

(a) centrifuging mouse whole blood for purification of platelet-rich plasma (PRP) to obtain platelets;

(b) adjusting the concentration of platelets with a buffer to 2.5 to 3×10⁸/mL;

(c) conducting a pre-activation process on the platelets using an agonist including ADP, collagen, thrombin, TRAP, ROS or combinations thereof to obtain the preactivated SC-PLT; and the transforming process of the SC-PLT is as follows: at the pre-activation process the rounded cell membrane is transformed into a pseudopodia form; at this time the preactivated SC-PLT is in a form of proaggregatory suspension particles, and the transmittance of the preactivated SC-PLT decreases to a stable state, and is negative in a blood agglutination, so as to obtain the SC-PLT; wherein when the platelets react with an agonist in a blood agglutination dosage, and then react with fibrinogen, the platelets irreversibly become a polymolecular procoagulant from a transition state of small suspension particles, and the transmittance of the platelets decreases rapidly and temporarily and then increases abruptly to the top (i.e., blood agglutination); wherein the SC-PLTs are preactivated disaggregated platelets, and the SC-PLTs emit biologically active substances stored in cells, α-granules, dense granules and lysosomes out to cells or transport these substances to the surface of cellular membrane; and the factors transported to the surface of cellular membrane include the cellular adhesion molecule;

(d) 5 min fixing the SC-PLTs with 0.5% paraformaldehyde and washing the released growth factors with a buffer, and suspending the SC-PLTs uniformly in the buffer. In one exemplary example the buffer is Tyrode's buffer.

### C. Experiments

(a) Animals: C57BL/6C (B6 albino) male mice are purchased from Charles River Taiwan Branch. The mice, 10 to 12 weeks of age, are fed with food and water ad libitum and are maintained on a 12-h light/dark cycle at 25±1° C. in an animal laboratory.

(b) Route of administration: Tyrode's buffer and SC-PLTs in a dosage of 1×10⁶, 1×10⁷, or 1×10⁸ are injected into mice respectively.

(c) Control group: sham operated mouse control group is used to monitor the surgical procedures, and the damage of sham operated mouse control group is closed to the relative baseline of the non-operated mouse control group.

(d) Statistics: the results are expressed as mean±SE or mean±SD, and P value <0.05 is considered statistically significant.

### D. Results

The invention provides a method of preparing a pharmaceutical composition of shape-changed platelets (SC-PLTs) for use in treating acute and emergent inflammatory injuries, comprising the steps of (a) preparing first, second, third, and fourth sets of mice wherein the first set of mice are for perfusion due to liver ischemia, the second set of mice are for observing dermal burn, the third set of mice are for observing sepsis, and the fourth set of mice are for local ischemia; (b) injecting 100-200 μL of Tyrode' buffer into each control mouse of the first set of mice; and injecting 100-200 μL of SC-PLTs in a first dosage of 1×10⁶, 1×10⁷, and 1×10⁸ cellular number into each sampling mouse of the first set of mice; (c) injecting 100-200 μL of Tyrode' buffer into each control mouse of the second set of mice; and injecting 100-200 μL of SC-PLTs in a second dosage of 1×10⁷, and 1×10⁸ cellular number into each sampling mouse of the second set of mice; (d) injecting 100-200 μL of Tyrode' buffer into each control mouse of the third set of mice; and injecting 100-200 μL of SC-PLTs in a third dosage of 1×10⁸ cellular number into each sampling mouse of the third set of mice; (e) injecting 100-200 μL of Tyrode' buffer into each control mouse of the fourth set of mice; and injecting 100-200 μL of SC-PLTs in a fourth dosage of 1×10⁸, and 2×10⁸ cellular number into each sampling mouse of the fourth set of mice; (f) observing healing of inflammatory injuries of the mice of each set of mice after a predetermine period of time; and (g) observing that the healing of inflammatory injuries of the mice of each set of mice is improved as the applied dosage of SC-PLTs in set 1, 2, 3 and 4; wherein the mice labeled C57BL/6C (B6 albino) are bought from Charles River Taiwan Branch, the mice are male, the mice are 10 to 12 weeks of age, the mice are kept in an animal laboratory at a temperature of 25±1° C., light cycle and dark cycle each are 12 hours per day, and food and water ad libitum are fed to the mice.

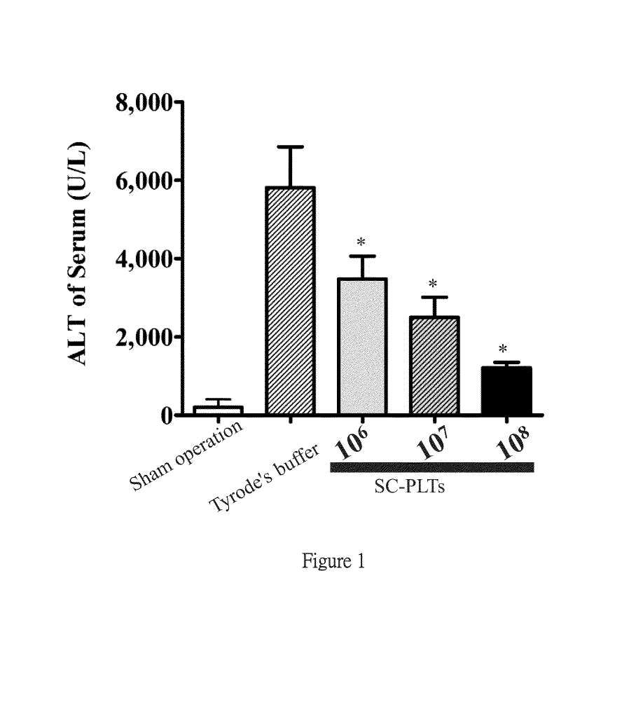

FIG. 1 is a diagram showing the effect of different dosages of SC-PLTs manipulation in mouse model of liver ischemia reperfusion:

C57BL/6c mice at age of 10-12 weeks are anesthetized by intraperitoneal (IP) injection of 100 mg/kg ketamine and 10 mg/kg xylazine and then subcutaneously injected with 50 μg/kg glycopyrrolate and 50 U/kg heparin to prevent excess salivation and possible suffocation. Eye lubricant is applied to prevent ocular dehydration. Mice are placed under a heating lamp and on a 37° C. heating pad. In some animals, rectal temperature is monitored with a TCAT-1A temperature control unit (Physitemp); it is found to be 35° C.±1° C. and did not differ between agent treatment groups. We used a partial hepatic ischemia model that spared the right lobe. This model avoids intestinal congestion, sepsis, and peritonitis, and it is not lethal during liver ischemia for up to 60 min Each mouse is placed in a supine position. A midline laparotomy and incision of the Linea Alba exposed the peritoneal cavity. The stomach and duodenum are displaced caudally to expose the hepatic triad and caudate lobes. The caudate lobe is separated gently from the left lobe and displaced from the right upper and lower lobes caudally to clearly view the hepatic triad above the bifurcation of right lobes, median lobe, and left lobe. A microaneurysm clip is applied to the hepatic triad above the bifurcation to clamp the flow of the hepatic artery, portal vein, and bile duct. The peritoneum is closed after superfusion with 200 μl of warm saline (37° C.). After 60 min of ischemia, the peritoneum is reopened, and the microaneurysm clip is removed Immediately before reperfusion is initiated, each mouse received either of a single bolus intravenous (IV) injection of SC-PLTs or vehicle control Tyrode's buffer. After 24 hr reperfusion, animals are reanesthetized, blood is obtained by cardiac puncture for serum alanine aminotransferase (ALT) activity assay, and liver is routinely removed for myeloperoxidase activity assay, Hematoxylin & Eosin (H&E) stain and immunohistochemistry assay. Here, we show the data of liver ALT assay by Means±SD. Tyrode's buffer and the SC-PLTs in a dosage of 1×10⁶, 1×10⁷, and 1×10⁸ respectively are injected into the jugular vein of liver ischemia mice after one hour of ischemia and immediately early reperfusion. The sham mice are performed by the same surgical procedure of open chest as experimental mice but without liver ischemia/reperfusion surgery and pharmacological treatment. The effect of reducing liver inflammatory injuries is positively correlated with the dosage of the SC-PLTs; that is, the SC-PLTs in a dosage of 1×10⁸ had the best therapeutic effect (n≧7, Mean±SD)*, p<0.001).

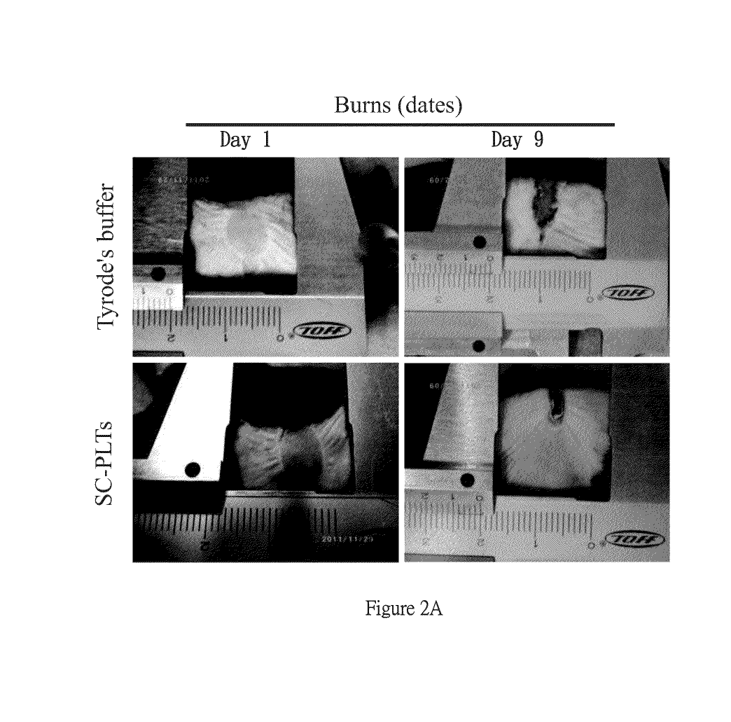

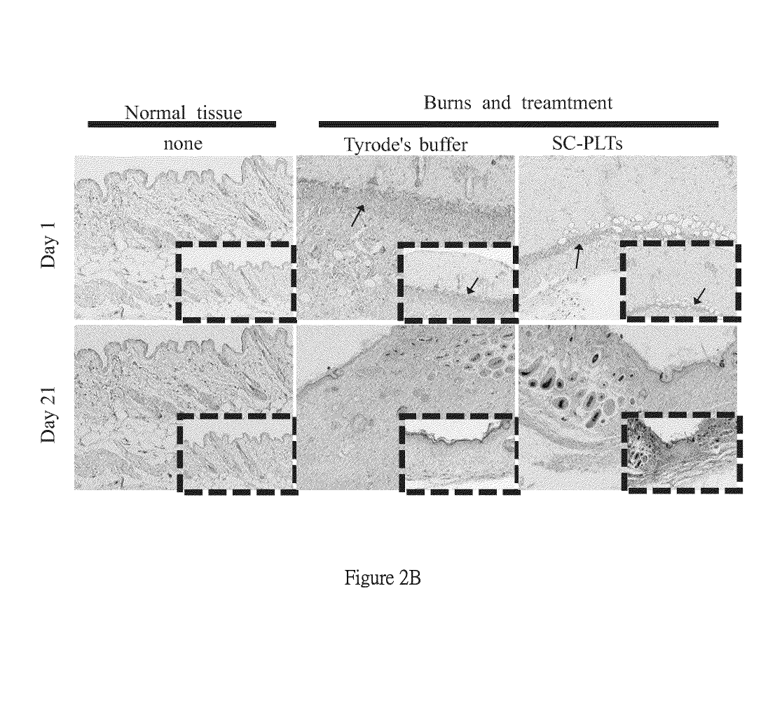

FIGS. 2A and 2B show the SC-PLTs used in dermal burn and repair. FIG. 2B shows the H&E stains of burns tissue treated with the SC-PLTs. The arrows indicate the aggregation of inflammatory cells, and the right bottom is the figure on a small scale. 8-12 weeks of C57BL/6c mice are anesthetized by ketamine (100 mg/kg) and xylazine (10 mg/kg) mixture using intraperitoneal injection. The dorsal hair is shaved by electronic clippers, and then applied depilatory to remove the residual hair. The dorsal skin is cleaned by saline and air dry, then lifted the dorsal skin by fingers and flatted skin by left palm with an interval of swab to prevent heat transduction from opposite side of lifted skin which is directly touched for 5 sec by using a cylindrical copper rod after the immersion of boiling water for 5 min 1×10⁸ SC-PLTs/200 μL tyrode's buffer are immediately injected through jugular vein, and then subcutaneously injected 1×10⁷ SC-PLTs/100 μL tyrode's buffer at dermal burn site. After immediate injection, SC-PLTs are injected daily for 5 consecutive days as above manipulation. A control group of C57BL/6 mice received with Tyrodes' buffer injection without SC-PLT content. Burn wound is determined at 0, 1 and 9 days by micrometer and photograph. After 1-day and 21-day treatment, skin tissues are cut and fixed in 18 volume ethanol: 7 volume FineFix (Milestone Srl, Sorisole, Torre Boldone, Italy) at 50° C. for 15-minute. Samples are then dehydrated in a grade series of solvent (70% ethanol at 4° C. for overnight, 100% ethanol at 65° C. for 25 min, 100% isopropanol at 70° C. for 1 h 55 min), and vaporization for 90 sec, and wax impregnation at 70° C. for 1 h 51 min) Samples are subsequently embedded in paraffin for trimmed and slice into 4-nm sections using a microtome (Leica RM 2135) with a TC65 tungsten knife (Thermo). Sections are deparaffinized and in changes of xylene and rehydrated in decreasing concentrations of ethanol. The sections are then subjected to H&E stain and viewed by light microscopy (Zeiss AxioSkop).

### H&E Stain

1. Heating samples at 60° C. for 30 mins.

2. Removing wax (100% xylene 5 mins=>100% xylene 5 mins=>100% ethanol 3 mins=>100% ethanol 3 mins=>95% ethanol 1 min=>95% ethanol 1 min=>80% ethanol 1 min=>soaking 3 mins in water)

3. Hematoxylin 1 min

4. Washing the samples for 3 mins 30 seconds in flowing tap water

5. 1% hydrocholoric acid 1 min

6. Washing the sample for 1 min in flowing tap water

7. 0.5% ammonium hydroxide 1 min

8. Washing the sample for 1 min in flowing tap water

9. Eosin 2 seconds

10. Washing the samples for 1 min in flowing tap water

11. Dehydration (95% ethanol 5 mins=>95% ethanol 5 mins=>100% ethanol 5 mins=>100% ethanol 5 mins=>100% xylene 5 mins=>100% xylene 5 mins)

12. Sealing the samples

The results proved that the use of SC-PLTs is far better than Tyrode's buffer in healing and repairing of dermal burn and reducing acute inflammatory injuries.

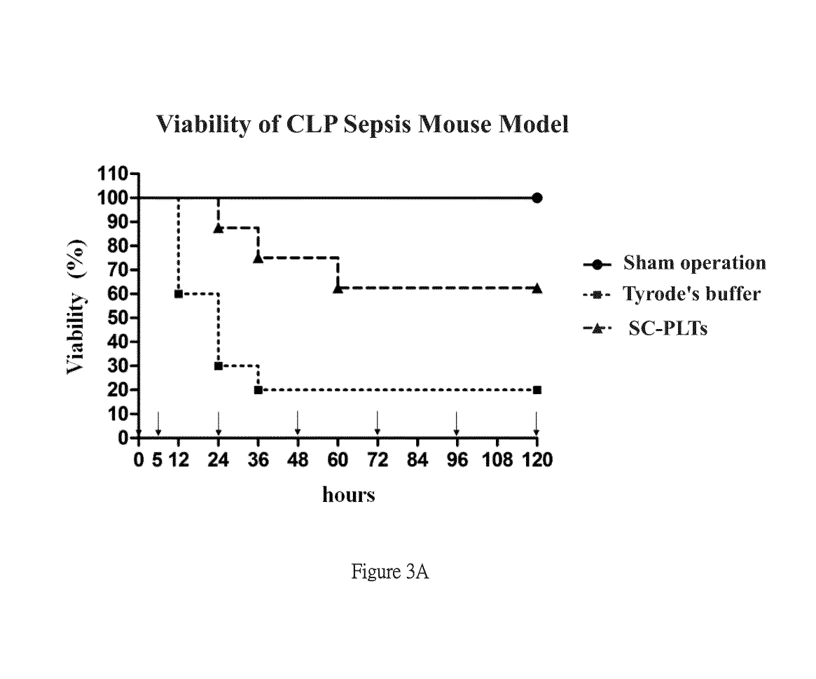

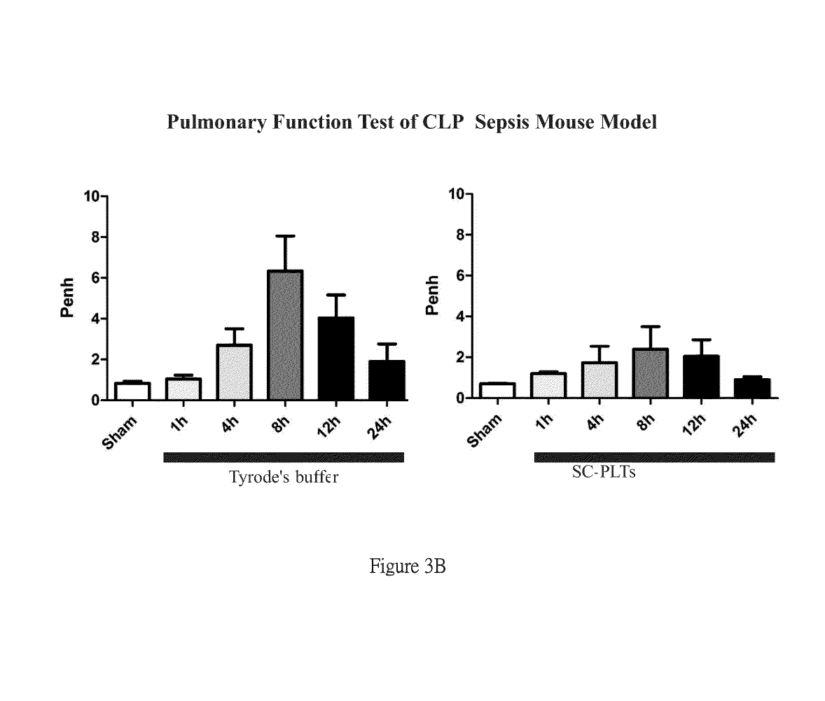

FIG. 3A shows the SC-PLTs used in reducing inflammatory injuries incurred by sepsis. After cecal ligation and puncture (CLP) operation, the mice are intravenously injected and in situ abdominal injected with the SC-PLTs (or Tyrode's buffer for the control group), and the same method and dosage are given as follows: a cellular dosage of 1×10⁸ SC-PLTs in 100 μL of Tyrode's buffer, are immediately subjected to intraperitoneal injection after the surgery of cecal ligation and puncture (CLP), and then boost another one injection of SC-PLTs via jaguar vein after intervals of 5 hours. For next 5 consecutive days, the SC-PLTs are simultaneously inject to peritoneal cavity and jaguar vein of septic mice by a dosage of 1×10⁸ dosage of SC-PLTs in 100 μL Tyrode's buffer. The injection time is indicated as the arrows at 0, 5, 24, 48, 72, 96, 120 hours. Intravenously injection of the SC-PLTs is in a dosage of 1×10⁸ SC-PLT/100 μL and in situ abdominal injected is in a dosage of 1×10⁷ SC-PLT/100 μL Mean±SD)*, p<0.05). The results indicated that the use of the SC-PLTs is far better than Tyrode's buffer in viability in sepsis.

SC-PLTs manipulation in mouse model of sepsis:

8-12 weeks male C57BL/6c mice are anesthetized by ketamine (100 mg/kg) and xylazine (10 mg/kg) mixture using intraperitoneal injection. Body temperature of animals are monitored by TH-8 Therm alert monitoring thermometer (Physitemp) and maintained at 36.5° C. by TCAT-1A temperature control. After midline laparotomy, the cecum is ligated, punctured, and then squeezed. Finally, the cecum is put back in the peritoneal cavity and the muscular and dermal wound are separately sutured with 4-0 black silk. The sham mice are performed by the surgery of laparotomy but without cecal ligation and puncture (CLP). After the CLP surgery, SC-PLTs or vehicle control Tyrode's buffer are immediately applied via intraperitoneal (IP) injection after intervals of 5 hr followed by intravenous (IV) injection jugular vein of SC-PLTs (1×10⁸/100 μL) or Tyrode's buffer. For next 5 consecutive days, SC-PLTs are injected daily by simultaneous IP and IV injection. Mice survival rate is determined as follows: 100 (mice survival numbers/mice total number) (%). In addition, pulmonary function is assessed by whole-body plethysmography as follows: Mice are placed unrestrained and unsedated in a cylindrical plethysmograph chamber (EMMS, Bordon, Hants) made of clear plastic. Mice are transferred directly from their cage into the chamber and allowed to acclimate to the chamber. Total 29 different parameters that can be generated by the machine, we focus on the measurements of Pause enhanced (Perth), Tidal Volume, Breathing Frequency, Minute Volume at a 10-min period after animal acclimatization. Here, we show the calculation of Penh by Means±SEM. The peritoneal lavage cells are harvested at 8 hr after CLP surgery as follows: Mouse is euthanized by CO₂, and sterilized abdomen by 70% ethanol spray. The outer skin of peritoneum is cross-cut and gently pull it back to expose the inner skin lining the peritoneal cavity. We injected 5 ml of prechilled DMEM (Gibco) containing 1 mM EDTA into the peritoneal cavity using a 22G needle. After injection, gently massage the peritoneum and shake mouse body to dislodge any attached cells. The peritoneal cells are harvested using a 22 G needle (bevel up) attached to a 5 ml syringe to suck peritoneal fluid, and deposit it in 50 mL conical tube (Falcon no. 2070) kept on ice after removing the needle from the syringe. The procedures of above mentioned peritoneal lavage are repeated twice by using 5 mL prechilled DMEM-1 mM EDTA. Then, the inner skin of the peritoneum is incised for exposing the peritoneal cavity. We collected the remaining fluid from cavity using pipette. All the peritoneal cells are pooled in a same 50 mL conical tube and pipetted the cell suspension to further dissociate cells, and then passed through a 40-nm cell strainer to remove the bigger cell aggregates. Then, cell suspension is washed twice in PBS by centrifugation (578×g for 5 min at 4° C.). Cell pellets are resuspended in flow cytometry staining buffer (eBioscience) and adjusted cell concentration to 5×10⁶/mL for 15 min incubation 0.1 ml aliquots are stained for 30 min in the dark, using specific antibodies-conjugated PE for anti-mouse CD11b, anti-mouse Ly6G, and anti-mouse CD3e. Stained cells are resuspended to 1 mL Dulbecco's phosphate buffered saline (PBS). The fluorescence intensity is measured by Beckman Coulter Cytomics FC500 Flow Cytometry with a minimum of 20,000 events.

FIG. 3B shows the SC-PLTs used in reducing complications of (lung) respiratory failure incurred by sepsis. Body box is used to measure mice breathing pattern (Penh) and to assess lung failure. The figure shows the curve of time and Pehn after the first dosage treatment given immediately after operation. The results prove that the use of the SC-PLTs reduced post-operation Penh index; that is, SC-PLTs can be used to reduce complications of (lung) respiratory failure incurred by sepsis.

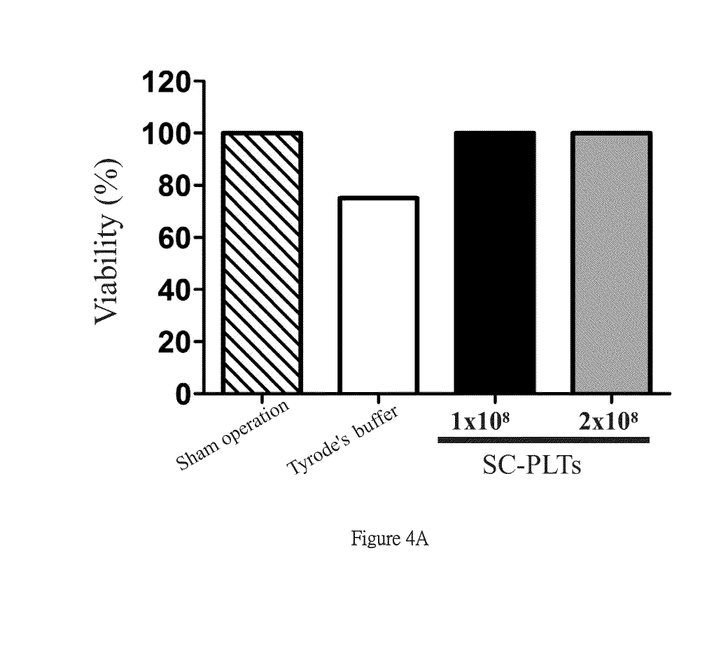

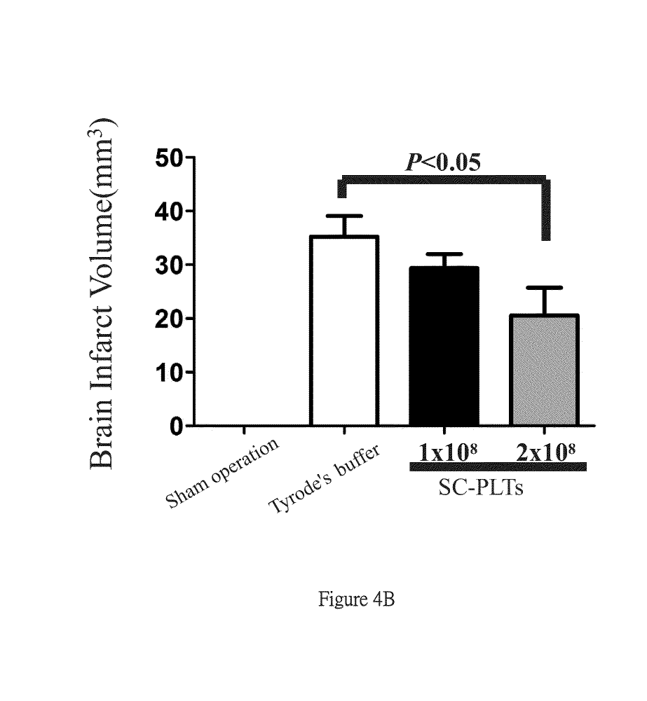

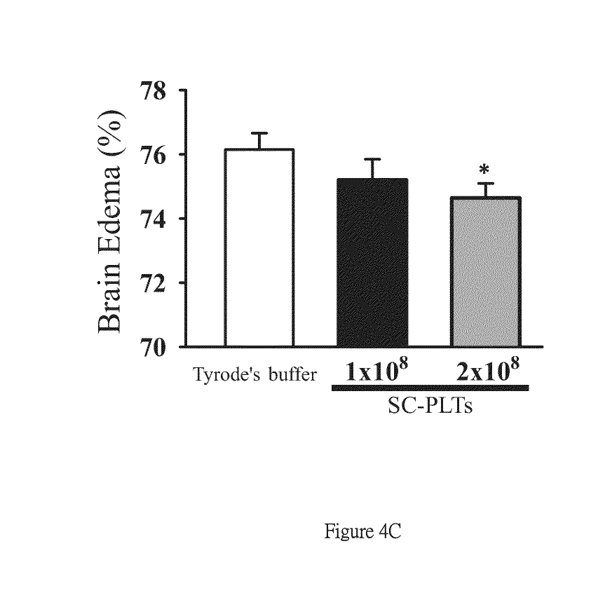

FIG. 4A is a schematic diagram showing the SC-PLTs used for reducing damage incurred by ischemic stroke. 8-week male C57BL/6c mice are anesthetized with chlorohydrate (450 mg/kg, intraperitoneal injection) and body temperature is maintained during surgery at 37±0.5° C. with a heating pad servo-controlled by a rectal probe. Focal ischemic infarcts are produced in the right lateral cerebral cortex in the territory of the middle cerebral artery (MCA). Both common carotid arteries are exposed by an anterior midline cervical incision. The animals are then placed in a lateral position, and a skin incision is made at the midpoint between the right lateral canthus and the anterior pinna. The temporal muscle is retracted, and a small craniotomy is made at the junction of the zygoma and squamosal bone using a drill (Dremel Multipro+5395, Dremel com. USA) cooled with saline solution. The dura is opened with fine forceps using a dissecting microscope (OPMI-1, ZISS, Germany), and the right MCA is permanent cut by monopolar electrosurgery. Both common carotid arteries are then occluded by microaneurysm clips for 20 min After removing the clips, 1×10⁸ dose and 2×10⁸ dose of SC-PLTs are respectively and immediately injected to ischemia stroke mice by jugular vein injection, and then repeated SC-PLTs treatment at 5 hr intervals. Twenty-four hours after cerebral infarction, the animals are anesthetized with chlorohydrate (450 mg/kg, intraperitoneal injection) and killed by rapid decapitation. Brains are removed, inspected visually for the anatomy of the MCA and for signs of hemorrhage or infection, immersed in cold saline solution, and sectioned into standard coronal slices using a brain matrix slicer (JACOBOWITZ Systems, Zivic-Miller Laboratories INC, Allison Park, USA). Slices are placed in the vital dye 2, 3, 5-triphenyltetrazolium chloride (TTC, 2%; Sigma, USA) at 37° C. and kept in the dark for 30 minutes, followed by 10% formalin at room temperature overnight. The outline of the right and left cerebral hemispheres as well as that of the infracted tissue, clearly visible by a lack of TTC staining, is outlined on the posterior surface of each slice using an image analyzer (color image scanner, EPSON GT-9000), connected to an image analysis system (AIS software, Imaging research INC, Canada) run on a personal computer, AMD K6-2 3D 400. Infarct volume is calculated as the sum of infarct area per slice multiplied by slice thickness. Both the surgeon and image analyzer operator are blinded to the treatment of each animal. In addition, brains of ischemia stroke are quickly removed 24 h after reperfusion, and the cerebral cortex and striatum are dissected and immediately put at 90° C. oven (Model: D0-45A; CHERNG CUEI CO., LTD. ROC) for 24 hours. Samples are assayed by the dry weight method as follows: Water content in samples is calculated as follows: 100 (wet wt−dry wt)/wet wt (%). The results indicate that the viability is higher in the SC-PLTs group than in the Tyrode's buffer group.

FIG. 4B shows the SC-PLTs used for reducing brain infarction incurred by ischemic stroke (n=8, Mean±SE)*, p<0.05. The results show that the brain infarction damage area is less in the SC-PLTs group than in the control group (Tyrode's buffer). Furthermore, using 2×10⁸ SC-PLTs is better than using 1×10⁸ SC-PLTs and SC-PLT/100 μL, in terms of the effect of reducing brain infarction.

FIG. 4C shows the SC-PLTs for reducing brain edema incurred by ischemic stroke, the brain water content (%) in the cerebral cortex and striatum (n=8, Mean±SE)*, p<0.05. The results show that the brain edema water content (%) incurred by ischemic stroke is lower in the SC-PLTs group than in the control group (Tyrode's buffer). Furthermore, using 2×10⁸ SC-PLTs is better than using 1×10⁸ SC-PLTs and SC-PLT/100 μL, in terms of mitigating brain edema.

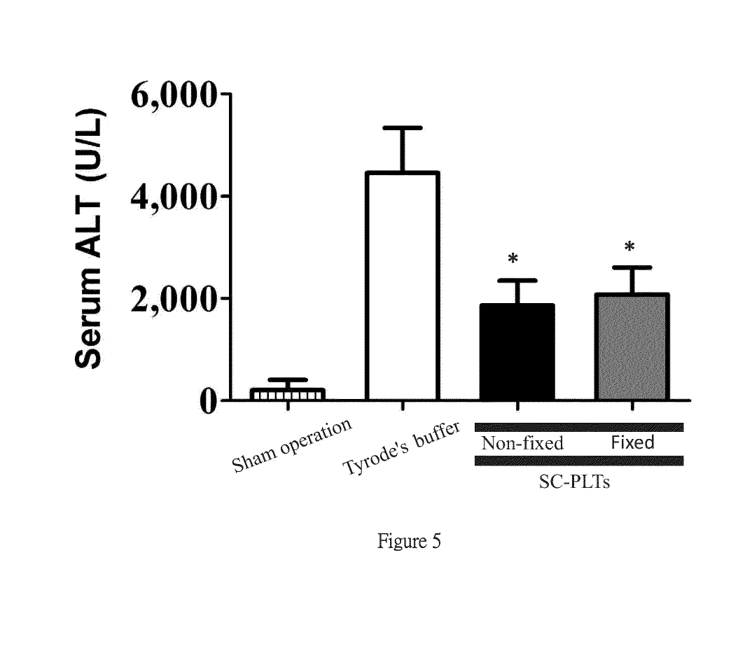

FIG. 5 shows the SC-PLTs and fixed-SC-PLTs used for reducing 60 minutes liver ischemia-reperfusion (n≧8, Mean±SD)*, p<0.05. The result shows that the SC-PLTs and fixed-SC-PLTs are better than Tyrode's buffer in reducing 60 minutes liver ischemia-reperfusion.

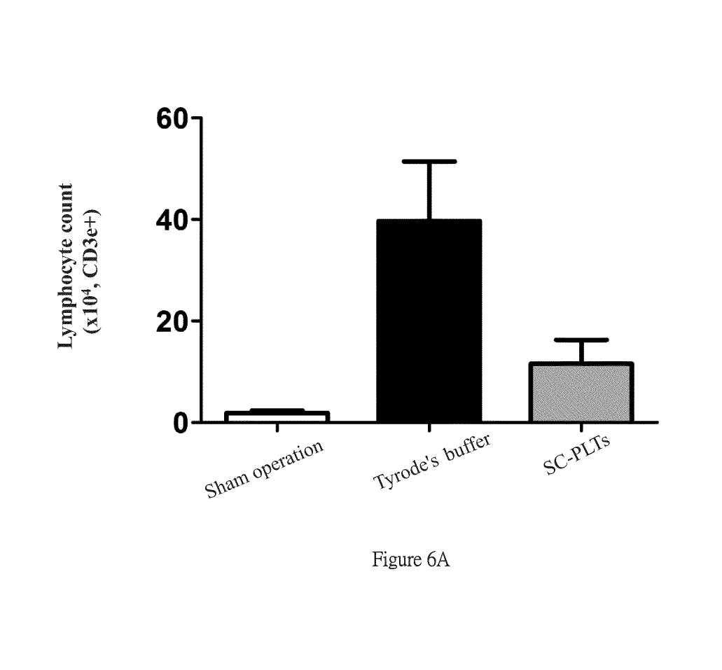

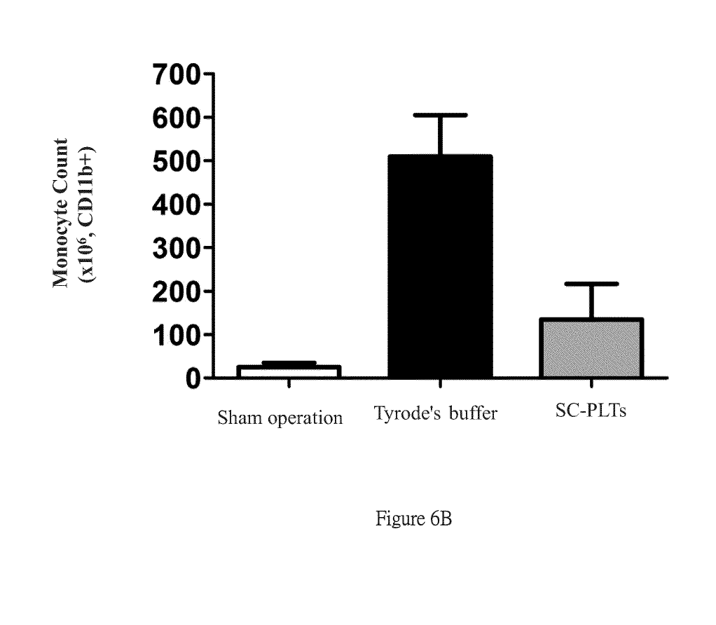

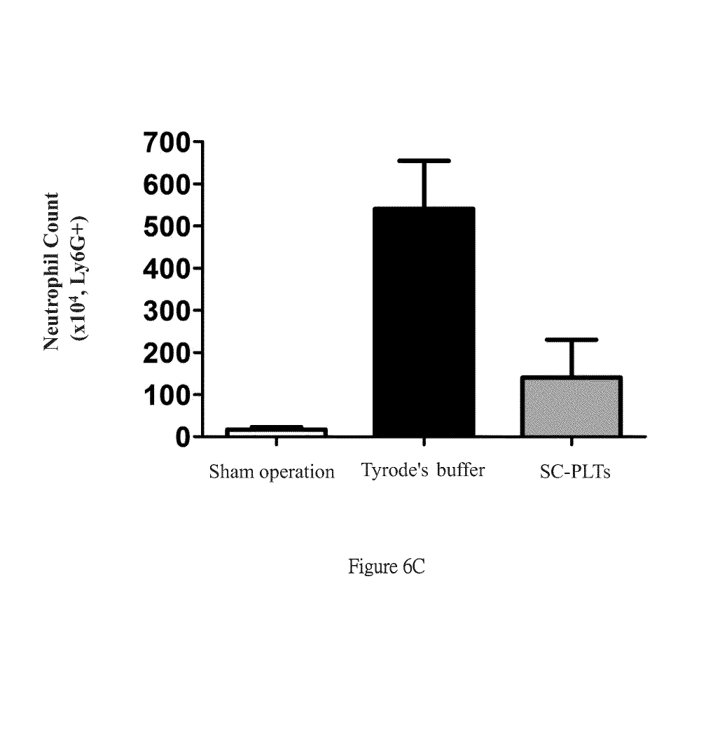

FIG. 6A shows that the SC-PLTs are better than Tyrode's buffer in lowering the abdominal inflammatory cells (CD3e+T lymphocyte) in the CLP sepsis mice.

FIG. 6B shows that the SC-PLTs are better than Tyrode's buffer in lowering abdominal CD 11b+monocyte in the CLP sepsis mice.

FIG. 6C shows that the SC-PLTs are better than Tyrode's buffer in lowering abdominal Ly6G+neutrophil in the CLP sepsis mice.

As showed in FIGS. 1, 4A, 4B, and 4C, comparing liver inflammatory injuries after 24 hours reperfusion, the results prove that the effect of the SC-PLTs is better than that of Tyrode's buffer; furthermore, the effect of 2×10⁸ SC-PLTs/200 μL is better than that of 1×10⁸ SC-PLTs/200 μL in ischemia stroke. Therefore, the effect of reducing acute inflammatory injuries is positively correlated to the injection dosage of the SC-PLTs.

While the invention has been described in terms of preferred embodiments, those skilled in the art will recognize that the invention can be practiced with modifications within the spirit and scope of the appended claims.

What is claimed is:

1. A method of preparing a pharmaceutical composition of shape-changed platelets (SC-PLTs) for use in treating acute and emergent inflammatory injuries, comprising the steps of:
(a) preparing first, second, third, and fourth sets of mice wherein the first set of mice are for perfusion due to liver ischemia, the second set of mice are for observing dermal burn, the third set of mice are for observing sepsis, and the fourth set of mice are for local ischemia;
(b) injecting 100-200 μL of Tyrode' buffer into each control mouse of the first set of mice; and injecting 100-200 μL of SC-PLTs in a first dosage of 1×10⁶, 1×10⁷, and 1×10⁸ cellular number into each sampling mouse of the first set of mice;
(c) injecting 100-200 μL of Tyrode' buffer into each control mouse of the second set of mice; and injecting 100-200 μL of SC-PLTs in a second dosage of 1×10⁷, and 1×10⁸ cellular number into each sampling mouse of the second set of mice;
(d) injecting 100-200 μL of Tyrode' buffer into each control mouse of the third set of mice; and injecting 100-200 μL of SC-PLTs in a third dosage of 1×10⁸ cellular number into each sampling mouse of the third set of mice;
(e) injecting 100-200 μL of Tyrode' buffer into each control mouse of the fourth set of mice; and injecting 100-200 μL of SC-PLTs in a fourth dosage of 1×10⁸, and 2×10⁸ cellular number into each sampling mouse of the fourth set of mice;
(f) observing healing of inflammatory injuries of the mice of each set of mice after a predetermine period of time; and
(g) observing that the healing of inflammatory injuries of the mice of each set of mice is improved as the dosage of SC-PLTs increases.

2. The method of preparing a pharmaceutical composition of SC-PLTs for use in treating acute and emergent inflammatory injuries of claim 1, wherein the mice are male, the mice are 10 to 12 weeks of age, the mice are kept in an animal laboratory at a temperature of 25±1° C., light cycle and dark cycle each are 12 hours per day, and food and water ad libitum are fed to the mice.

3. The SC-PLT as claimed in claim 1, wherein the acute and emergent inflammatory disease is mitigated by activating and transforming the SC-PLT to release cellular adhesion molecules, and the cellular adhesion molecules transfer to the surface of the SC-PLT; the SC-PLT containing the cellular adhesion molecules is able to adsorb stromal vascularity inflammatory immune cells from inflamed and damaged place, and the inflammatory immune cells (inflammatory leukocyte) are eliminated through the circulatory system; and the SC-PLT is able to decrease the concentration of inflammation-related cells including neutrophil, monocyte, and lymphocyte in blood.

4. The method of preparing a pharmaceutical composition of SC-PLTs for use in treating acute and emergent inflammatory injuries of claim 1, wherein the SC-PLTs are prepared by the steps of:
(a) male mice are anesthetized and blood is obtained by cardiac puncture, blood is collected into a sterile tube containing acid citrate dextrose (ACD) by ratio of 6:1, and then centrifuged at 25° C. and at a maximum speed of 2,300×g for 8 sec per ml, acceleration and brake speed of the centrifugation are set to 9 and 0, respectively, the upper layer containing platelet-rich plasma (PRP) is harvested to another sterile tube, and then subjected to centrifugation at 2,000×g for 3 min per ml, discard the supernatant, and redissolve pellets in 5 ml Tyrode's albumin buffer containing 10 U/mL heparin, and 0.5 μM prostacyclin, after 10-min incubation at 37° C. and centrifugation, cell pellets are resuspended in 5 ml Tyrode's albumin buffer containing 0.5 μM Prostaglandin 12 (PGI2);
(b) platelet number is counted by an analyzer and adjusted to a concentration of 2.5˜3×10⁸ platelets/ml with Tyrode's albumin buffer containing 0.02 U/ml apyrase, after 1˜2 min incubation at 25° C., centrifugation, and wash once, the cell pellets are resuspended into Tyrode's albumin buffer; Platelets are activated by thrombin, and tracked the transition of shape-change platelets (SC-PLTs) by platelet aggregometry, the platelet solution is immediately centrifuged, and then pellets are washed, and then resupended in Tyrode's albumin buffer; and
(c) conducting a pre-activation process on the platelets using an agonist including adenosine diphosphate (ADP), collagen, thrombin, thrombin receptor agonist peptide (TRAP), reactive oxygen species (ROS) or combinations thereof to obtain the preactivated SC-PLT; and the transforming process of the SC-PLT is as follows: at the pre-activation process the rounded cell membrane is transformed into a pseudopodia form; at this time the preactivated SC-PLT is in a form of proaggregatory suspension particles, and the transmittance of the preactivated SC-PLT decreases to a stable state, and is negative in a blood agglutination, so as to obtain the SC-PLT; wherein when the platelets react with an agonist in a blood agglutination dosage, and then react with fibrinogen, the platelets irreversibly become a polymolecular procoagulant from a transition state of small suspension particles, and the transmittance of the platelets in decreases rapidly and temporarily and then increases abruptly to the top;
wherein the SC-PLTs are preactivated disaggregated platelets, and the SC-PLTs emit biologically active substances stored in cells, α-granules, dense granules and lysosomes out to cells or transport these substances to the surface of cellular membrane; and the factors transported to the surface of cellular membrane include the cellular adhesion molecule.

5. A method of preparing a pharmaceutical composition of shape-changed platelets (SC-PLTs) for use in treating acute and emergent inflammatory injuries, comprising the steps of:
(a) preparing first, second, third, and fourth sets of mice wherein the first set of mice are for perfusion due to liver ischemia, the second set of mice are for observing dermal burn, the third set of mice are for observing sepsis, and the fourth set of mice are for local ischemia;
(b) injecting 100-200 μL of Tyrode' buffer into each control mouse of the first set of mice; and injecting 100-200 μL of SC-PLTs in a first dosage of 1×10⁶, 1×10⁷, and 1×10⁸ cellular number into each sampling mouse of the first set of mice;
(c) injecting 100-200 μL of Tyrode' buffer into each control mouse of the second set of mice; and injecting 100-200 μL of SC-PLTs in a second dosage of 1×10⁷, and 1×10⁸ cellular number into each sampling mouse of the second set of mice;
(d) injecting 100-200 μL of Tyrode' buffer into each control mouse of the third set of mice; and injecting 100-200 μL of SC-PLTs in a third dosage of 1×10⁸ cellular number into each sampling mouse of the third set of mice;
(e) injecting 100-200 μL of Tyrode' buffer into each control mouse of the fourth set of mice; and injecting 100-200 μL of SC-PLTs in a fourth dosage of 1×10⁸, and 2×10⁸ cellular number into each sampling mouse of the fourth set of mice;
(f) observing healing of inflammatory injuries of the mice of each set of mice after a predetermine period of time;
wherein the mice of the first set of mice are C57BL/6c mice at age of 10-12 weeks are anesthetized by IP injection of 100 mg/kg ketamine and 10 mg/kg xylazine and then subcutaneously injected with 50 μg/kg glycopyrrolate and 50 U/kg heparin to prevent excess salivation and possible suffocation;
wherein eye lubricant is applied to prevent ocular dehydration;
wherein the mice are placed under a heating lamp and on a 37° C. heating pad;
wherein rectal temperature 35° C.±1° C. is monitored with a TCAT-1A temperature control unit;
wherein a partial hepatic ischemia model is used for avoiding intestinal congestion, sepsis, and peritonitis, and it is not lethal during liver ischemia for up to 60 min;
wherein each mouse is placed in a supine position;
wherein a midline laparotomy and incision of the Linea Alba is exposed the peritoneal cavity;
wherein the stomach and duodenum are displaced caudally to expose the hepatic triad and caudate lobes;
wherein the caudate lobe is separated from the left lobe and displaced from the right upper and lower lobes caudally to clearly view the hepatic triad above the bifurcation of right lobes, median lobe, and left lobe;
wherein a microaneurysm clip is applied to the hepatic triad above the bifurcation to clamp the flow of the hepatic artery, portal vein, and bile duct;
wherein the peritoneum is closed after superfusion with 200 μl of warm saline at 37° C.;
wherein after 60 min of ischemia, the peritoneum is reopened, and the microaneurysm clip is removed;
wherein immediately before reperfusion is initiated, each mouse receives either a single bolus IV injection of SC-PLTs at 1×10⁸ dose or vehicle control Tyrode's buffer; and
wherein after 24 hr reperfusion, animals are reanesthetized, blood is obtained by cardiac puncture for serum alanine aminotransferase (ALT) activity assay, and liver is routinely removed for myeloperoxidase activity assay, Hematoxylin & Eosin (H&E) stain and immunohistochemistry assay.

6. A method of preparing a pharmaceutical composition of shape-changed platelets (SC-PLTs) for use in treating acute and emergent inflammatory injuries, comprising the steps of:
(a) preparing first, second, third, and fourth sets of mice wherein the first set of mice are for perfusion due to liver ischemia, the second set of mice are for observing dermal burn, the third set of mice are for observing sepsis, and the fourth set of mice are for local ischemia;
(b) injecting 100-200 μL of Tyrode' buffer into each control mouse of the first set of mice;
and injecting 100-200 μL of SC-PLTs in a first dosage of 1×10⁶, 1×10⁷, and 1×10⁸ cellular number into each sampling mouse of the first set of mice;
(c) injecting 100-200 μL of Tyrode' buffer into each control mouse of the second set of mice; and injecting 100-200 μL of SC-PLTs in a second dosage of 1×10⁷, and 1×10⁸ cellular number into each sampling mouse of the second set of mice;
(d) injecting 100-200 μL of Tyrode' buffer into each control mouse of the third set of mice; and injecting 100-200 μL of SC-PLTs in a third dosage of 1×10⁸ cellular number into each sampling mouse of the third set of mice;
(e) injecting 100-200 μL of Tyrode' buffer into each control mouse of the fourth set of mice; and injecting 100-200 μL of SC-PLTs in a fourth dosage of 1×10⁸, and 2×10⁸ cellular number into each sampling mouse of the fourth set of mice;
(f) observing healing of inflammatory injuries of the mice of each set of mice after a predetermine period of time;
wherein the mice of the second set of mice are C57BL/6c mice at age of 10-12 weeks are anesthetized by ketamine (100 mg/kg) and xylazine (10 mg/kg) mixture using intraperitoneal injection;
wherein the dorsal hair is shaved by electronic clippers, and then applied depilatory to remove the residual hair;
wherein the dorsal skin is cleaned by saline and air dry, then lifted the dorsal skin by fingers and flatted skin by left palm with an interval of swab to prevent heat transduction from opposite side of lifted skin which is directly touched for 5 sec by using a cylindrical copper rod after the immersion of boiling water for 5 min;
wherein 1×10⁸ SC-PLTs/200 μL tyrode's buffer are immediately injected through jugular vein, and then subcutaneously injected 1×10⁷ SC-PLTs/100 μL tyrode's buffer at dermal burn site;
wherein after immediate injection, SC-PLTs are injected daily for 5 consecutive days as above manipulation;
wherein a control group of C57BL/6 mice receive with Tyrodes' buffer injection without SC-PLT content;
wherein burn wound is determined at 0, 1 and 9 days by micrometer and photograph;
wherein after 1-day and 21-day treatment, skin tissues are cut and fixed in 18 volume ethanol: 7 volume FineFix at 50° C. for 15-minute;
wherein samples are then dehydrated in a grade series of solvent having 70% ethanol at 4° C. for overnight, 100% ethanol at 65° C. for 25 min, and 100% isopropanol at 70° C. for 1 h 55 min, and vaporization for 90 sec, and wax impregnation at 70° C. for 1 h 51 min;
wherein the samples are subsequently embedded in paraffin for trimmed and slice into 4-nm sections using a microtome with a TC65 tungsten knife;
wherein sections are deparaffinized and in changes of xylene and rehydrated in decreasing concentrations of ethanol; and
wherein the sections are then subjected to H&E stain and viewed by light microscopy.

7. A method of preparing a pharmaceutical composition of shape-changed platelets (SC-PLTs) for use in treating acute and emergent inflammatory injuries, comprising the steps of:
(a) preparing first, second, third, and fourth sets of mice wherein the first set of mice are for perfusion due to liver ischemia, the second set of mice are for observing dermal burn, the third set of mice are for observing sepsis, and the fourth set of mice are for local ischemia;
(b) injecting 100-200 μL of Tyrode' buffer into each control mouse of the first set of mice; and injecting 100-200 μL of SC-PLTs in a first dosage of 1×10⁶, 1×10⁷, and 1×10⁸ cellular number into each sampling mouse of the first set of mice;
(c) injecting 100-200 μL of Tyrode' buffer into each control mouse of the second set of mice; and injecting 100-200 μL of SC-PLTs in a second dosage of 1×10⁷, and 1×10⁸ cellular number into each sampling mouse of the second set of mice;
(d) injecting 100-200 μL of Tyrode' buffer into each control mouse of the third set of mice; and injecting 100-200 μL of SC-PLTs in a third dosage of 1×10⁸ cellular number into each sampling mouse of the third set of mice;
(e) injecting 100-200 μL of Tyrode' buffer into each control mouse of the fourth set of mice; and injecting 100-200 μL of SC-PLTs in a fourth dosage of 1×10⁸, and 2×10⁸ cellular number into each sampling mouse of the fourth set of mice; and
(f) observing healing of inflammatory injuries of the mice of each set of mice after a predetermine period of time;
wherein the mice of the third set are 8-12 weeks male C57BL/6c mice and anesthetized by ketamine of 100 mg/kg and xylazine of 10 mg/kg mixture using intraperitoneal injection;
wherein body temperature of animals are monitored by TH-8 Therm alert monitoring thermometer and maintained at 36.5° C. by TCAT-1A temperature control;
wherein after midline laparotomy, the cecum is ligated, punctured, and then squeezed;
wherein finally, the cecum is put back in the peritoneal cavity and the muscular and dermal wound are separately sutured with 4-0 black silk;
wherein the sham mice are performed by the surgery of laparotomy but without cecal ligation and puncture (CLP);
wherein after the CLP surgery, SC-PLTs or vehicle control Tyrode's buffer are immediately applied via intraperitoneal (IP) injection after intervals of 5 hr followed by intravenous (IV) injection jugular vein of SC-PLTs (1×10⁸/100 μL) or Tyrode's buffer;
wherein in next 5 consecutive days, SC-PLTs are injected daily by simultaneous IP and IV injection;
wherein mice survival rate is determined as follows: 100 (mice survival numbers/mice total number) (%);
wherein pulmonary function is assessed by whole-body plethysmography as follows: Mice are placed unrestrained and unsedated in a cylindrical plethysmograph chamber;
wherein mice are transferred directly from their cage into the chamber and allowed to acclimate to the chamber;
wherein total 29 different parameters are generated by the machine, we focus on the measurements of Pause enhanced (Penh), Tidal Volume, Breathing Frequency, Minute Volume at a 10-min period after animal acclimatization;
wherein the calculation of Penh by Means±SEM is shown;
wherein the peritoneal lavage cells are harvested at 8 hr after CLP surgery as follows: Mouse is euthanized by CO₂ and sterilized abdomen by 70% ethanol spray;
wherein the outer skin of peritoneum is cross-cut and gently pull it back to expose the inner skin lining the peritoneal cavity;
wherein 5 ml of prechilled DMEM containing 1 mM EDTA is injected into the peritoneal cavity using a 22G needle;
wherein after injection, gently massage the peritoneum and shake mouse body to dislodge any attached cells;
wherein the peritoneal cells are harvested using a 22 G needle attached to a 5 ml syringe to suck peritoneal fluid, and deposit it in 50 mL conical tube kept on ice after removing the needle from the syringe;
wherein the procedures of above mentioned peritoneal lavage are repeated twice by using 5 mL prechilled DMEM-1 mM EDTA;
wherein the inner skin of the peritoneum is incised for exposing the peritoneal cavity;
wherein the remaining fluid from cavity is collected using pipette;
wherein all the peritoneal cells are pooled in a same 50 mL conical tube and pipetted the cell suspension to further dissociate cells, and then passed through a 40-nm cell strainer to remove the bigger cell aggregates;
wherein cell suspension are washed twice in PBS by centrifugation of 578×g for 5 min at 4° C.;
wherein cell pellets are resuspended in flow cytometry staining buffer and adjusted cell concentration to 5×10⁶/mL for 15 min and incubation 0.1 ml aliquots are stained for 30 min in the dark using specific antibodies-conjugated PE for anti-mouse CD11b, anti-mouse Ly6G, and anti-mouse CD3e;
wherein stained cells are resuspended to 1 mL Dulbecco's phosphate buffered saline (PBS); and
wherein the fluorescence intensity is measured by a flow cytometry with a minimum of 20,000 events.

8. A method of preparing a pharmaceutical composition of shape-changed platelets (SC-PLTs) for use in treating acute and emergent inflammatory injuries, comprising the steps of:
(a) preparing first, second, third, and fourth sets of mice wherein the first set of mice are for perfusion due to liver ischemia, the second set of mice are for observing dermal burn, the third set of mice are for observing sepsis, and the fourth set of mice are for local ischemia;
(b) injecting 100-200 μL of Tyrode' buffer into each control mouse of the first set of mice; and injecting 100-200 μL of SC-PLTs in a first dosage of 1×10⁶, 1×10⁷, and 1×10⁸ cellular number into each sampling mouse of the first set of mice;
(c) injecting 100-200 μL of Tyrode' buffer into each control mouse of the second set of mice; and injecting 100-200 μL of SC-PLTs in a second dosage of 1×10⁷, and 1×10⁸ cellular number into each sampling mouse of the second set of mice;
(d) injecting 100-200 μL of Tyrode' buffer into each control mouse of the third set of mice; and injecting 100-200 μL of SC-PLTs in a third dosage of 1×10⁸ cellular number into each sampling mouse of the third set of mice;
(e) injecting 100-200 μL of Tyrode' buffer into each control mouse of the fourth set of mice; and injecting 100-200 μL of SC-PLTs in a fourth dosage of 1×10⁸, and 2×10⁸ cellular number into each sampling mouse of the fourth set of mice; and
(f) observing healing of inflammatory injuries of the mice of each set of mice after a predetermine period of time;
wherein mice of the fourth set of mice are 8-week male C57BL/6c mice and anesthetized with chlorohydrate of 450 mg/kg, IP injection and body temperature is maintained during surgery at 37±0.5° C. with a heating pad servo-controlled by a rectal probe;
wherein focal ischemic infarcts are produced in the right lateral cerebral cortex in the territory of the middle cerebral artery (MCA);
wherein both common carotid arteries are exposed by an anterior midline cervical incision;
wherein the animals are then placed in a lateral position, and a skin incision is made at the midpoint between the right lateral canthus and the anterior pinna;
wherein the temporal muscle is retracted, and a small craniotomy is made at the junction of the zygoma and squamosal bone using a drill cooled with saline solution;
wherein the dura is opened with fine forceps using a dissecting microscope, and the right MCA is permanent cut by monopolar electrosurgery;
wherein both common carotid arteries are then occluded by microaneurysm clips for 20 min;
wherein after removing the clips, 1×10⁸ dose and 2×10⁸ dose SC-PLTs are respectively and immediately injected to ischemia stroke mice by jugular vein injection, and then repeated SC-PLTs treatment at 5 hr intervals;
wherein twenty-four hours after cerebral infarction, the animals are anesthetized with chlorohydrate of 450 mg/kg, IP injection and killed by rapid decapitation;
wherein brains are removed, inspected visually for the anatomy of the MCA and for signs of hemorrhage or infection, immersed in cold saline solution, and sectioned into standard coronal slices using a brain matrix slicer;
wherein slices are placed in the vital dye 2, 3, 5-triphenyltetrazolium chloride at 37° C. and kept in the dark for 30 minutes, followed by 10% formalin at room temperature overnight;
wherein the outline of the right and left cerebral hemispheres as well as that of the infracted tissue, clearly visible by a lack of TTC staining, is outlined on the posterior surface of each slice using an image analyzer, connected to an image analysis system run on a personal computer;
wherein infarct volume is calculated as the sum of infarct area per slice multiplied by slice thickness;
wherein both the surgeon and image analyzer operator are blinded to the treatment of each animal;
wherein brains of ischemia stroke are quickly removed 24 h after reperfusion, and the cerebral cortex and striatum are dissected and immediately put at 90° C. oven for 24 hours;
wherein samples are assayed by the dry weight method as follows: Water content in samples is calculated as follows: 100 (wet wt−dry wt)/wet wt (%).
